SEQUENCE LISTING (7 sequences)

SEQ ID NO: 1 - DNA, 2343 bp, Homo sapiens
atggagcggctttggggtctattccagagagcgcaacaactgtccccaagatcctctcagaccgtctaccagcgtgtggaaggcccccggaaagggcacctggaggaggaagaggaagacggggaggagggggcggagacattggcccacttctgccccatggagctgaggggccctgagcccctgggctctagacccaggcagccaaacctcattccctgggcggcagcaggacggagggctgccccctacctggtcctgacggccctgctgatcttcactggggccttcctactgggctacgtcgccttccgagggtcctgccaggcgtgcggagactctgtgttggtggtcagtgaggatgtcaactatgagcctgacctggatttccaccagggcagactctactggagcgacctccaggccatgttcctgcagttcctgggggaggggcgcctggaggacaccatcaggcaaaccagccttcgggaacgggtggcaggctcggccgggatggccgctctgactcaggacattcgcgcggcgctctcccgccagaagctggaccacgtgtggaccgacacgcactacgtggggctgcaattcccggatccggctcaccccaacaccctgcactgggtcgatgaggccgggaaggtcggagagcagctgccgctggaggaccctgacgtctactgcccctacagcgccatcggcaacgtcacgggagagctggtgtacgcccactacgggcggcccgaagacctgcaggacctgcgggccaggggcgtggatccagtgggccgcctgctgctggtgcgcgtgggggtgatcagcttcgcccagaaggtgaccaatgctcaggacttcggggctcaaggagtgctcatatacccagagccagcggacttctcccaggacccacccaagccaagcctgtccagccagcaggcagtgtatggacatgtgcacctgggaactggagacccctacacacctggcttcccttccttcaatcaaacccagaagctcaaaggccctgtggccccccaagaatggcaggggagcctcctaggctccccttatcacctgggccccgggccacgactgcggctagtggtcaacaatcacaggacctccacccccatcaacaacatcttcggctgcatcgaaggccgctcagagccagatcactacgttgtcatcggggcccagagggatgcatggggcccaggagcagctaaatccgctgtggggacggctatactcctggagctggtgcggaccttttcctccatggtgagcaacggcttccggccccgcagaagtctcctcttcatcagctgggacggtggtgactttggaagcgtgggctccacggagtggctagagggctacctcagcgtgctgcacctcaaagccgtagtgtacgtgagcctggacaacgcagtgctgggggatgacaagtttcatgccaagaccagcccccttctgacaagtctcattgagagtgtcctgaagcaggtggattctcccaaccacagtgggcagactctctatgaacaggtggtgttcaccaatcccagctgggatgctgaggtgatccggcccctacccatggacagcagtgcctattccttcacggcctttgtgggagtccctgccgtcgagttctcctttatggaggacgaccaggcctacccattcctgcacacaaaggaggacacttatgagaacctgcataaggtgctgcaaggccgcctgcccgccgtggcccaggccgtggcccagctcgcagggcagctcctcatccggctcagccacgatcgcctgctgcccctcgacttcggccgctacggggacgtcgtcctcaggcacatcgggaacctcaacgagttctctggggacctcaaggcccgcgggctgaccctgcagtgggtgtactcggcgcggggggactacatccgggcggcggaaaagctgcggcaggagatctacagctcggaggagagagacgagcgactgacacgcatgtacaacgtgcgcataatgcggatccccctctctgcgcaggtggagttctacttcctttcccagtacgtgtcgccagccgactccccgttccgccacatcttcatgggccgtggagaccacacgctgggcgccctgctggaccacctgcggctgctgcgctccaacagctccgggacccccggggccacctcctccactggcttccaggagagccgtttccggcgtcagctagccctgctcacctggacgctgcaaggggcagccaatgcgcttagcggggatgtctggaacattgataacaacttctga

SEQ ID NO: 2 - DNA, 45 bp, Homo sapiens
cccgccgtggcccaggccgtggcccagctcgcagggcagctcctc

SEQ ID NO: 3 - DNA, 33 bp, Homo sapiens
cccgccgtggcccagctcgcagggcagctcctc

SEQ ID NO: 4 - protein, 15 aa, Homo sapiens
PAVAQAVAQLAGQLL

SEQ ID NO: 5 - protein, 11 aa, Homo sapiens
PAVAQLAGQLL

SEQ ID NO: 6 - DNA, 23 bp, Homo sapiens
cccagcgtccaccctgtcctggc

SEQ ID NO: 7 - DNA, 20 bp, Homo sapiens
ctggattgccagagaggacc

(19) United States
(12) Patent Application Publication
(10) Pub. No.: US 2004/0033513 A1
(43) Pub. Date: Feb. 19, 2004

(54) Method and probes for the genetic diagnosis of hereditary haemocromatosis

(76) Inventors: Clara Camaschella, Torino (IT); Friedrich Kury, Wien (AT); Christian Oberkanins, Wien (AT)

Correspondence Address: THE FIRM OF KARL F ROSS, 5676 RIVERDALE AVENUE, PO BOX 900, RIVERDALE (BRONX), NY, 10471-0900, US

(21) Appl. No.: 10/399,385

(22) PCT Filed: Aug. 19, 2002
(86) PCT No.: PCT/AT02/00250

(30) Foreign Application Priority Data
Aug. 17, 2001 (AT) A 1295/01

(51) Int. Cl. C12Q 1/68

(52) U.S. Cl. 435/6

(57) ABSTRACT

In a diagnosis method for haemochromatosis, a biological sample is analyzed according to the invention for the presence of the nucleotide sequence

5′-cccgccgtggcccagctcgcagggcagctcctc-3′ (Sequence No. 3) instead of the nucleotide sequence 5′-cccgccgtggcccaggccgtggcccagctcgcagggcagctcctc-3′ (Sequence No. 2) based upon a 12 nucleotide long deletion in Exon 16 of the TFR2 cDNA sequence. Or according to the invention a biological sample is analyzed for the presence of nucleic acids which code for a TFR2 product with an amino acid sequence

Pro Ala Val Ala Gin Leu Ala Gly Gin Leu Leu (Sequence No. 5) instead of the amino sequence Pro Ala Val Ala Gin Ala Val Ala Gin Leu Ala Gly Gin Leu Leu (Sequence No. 4). A probe for the diagnosis of haemochromatosis is according to the invention capable of hybridization with nucleic acids of a biological sample in a region which contains the nucleotide sequence 5′-cccgccgtggcccagctcgcagggcagctcctc-3′ (Sequence No. 3) in Exon 16 of TFR2 cDNA sequence.

The invention relates to a method for the genetic diagnosis of hereditary haemochromatosis (HH) as well as to probes for the genetic diagnosis of hereditary haemochromatosis.

Haemochromatosis is a disturbance in the iron metabolism in the human body which is characterized by an iron overload. The excess iron deposits in a plurality of organs and gives rise to irreversible damage and illnesses resulting therefrom. Since the clinical symptoms of haemochromatosis often arise first at the ages of 40, 50 or more years and then are present already as irreversible organ damage, there are many demands for a presymptomatic diagnosis. This is even more important since, by regular venisection, the iron taken up in surplus can be simply removed.

In conjunction with haemochromatosis, numerous mutations have hitherto been found in the HFE gene (chromosome 6p, HH Type 1) (Feder et al, A novel MHC class 1-like gene is mutated in patients with hereditary haemochromatosis, Nat. Genet. 1996, 13:399-408; Villiers et al Spectrum of mutations in the HFE gene implicated in haemochromatosis and porphyria, Hum. Mol. Genet. 1999, 8:1517-22). The proof of these mutations in biological samples can be used for the genetic diagnosis of haemochromatosis.

A known haemochromatosis illness, which does not depend upon mutation in the HFE genes is juvenile haemochromatosis (HH Type 2). Its origin is localized on the chromosome 1q.

Recently, mutations also in the TFR2 Gene (chromosome 7q22, HH type 3) have been found and are associated with haemochromatosis. They are described together with further new mutations in the HFE gene in the international application PCT/EP01/04835 (WO 01/83812).

It is the object of the present invention to provide further possibilities of the finding genetic origins for haemochromatosis for diagnostics and to develop investigation techniques which can research previously known genetic origins more completely and to provide more reliable investigation results.

The object is achieved in that a biological sample is analyzed for the presence of the nucleotide sequence 5′cccgccgtggcccagctcgcagggcagctcctc3′ (Sequence No. 3) instead of nucleotide sequence 5′cccgccgtggcccaggccgtggcccagctcgcagggcagctcctc3′ (Sequence No. 2) by reason of a 12 nucleotide long deletion in Exon 16 of the TFR2 cDNA-sequence.

Especially, according to the invention the sample is analyzed for a deletion of the nucleotides 1780-1791 in Exon 16 of TFR2-α cDNA sequence. Reference to the TFR2-α cDNA sequence is a reference to the gene bank access number NM_{—}003227.1 and the associated sequence protocol (Sequence No. 1).

This mutation has been found in three siblings with haemochromatosis illness which have no presentation of the known HFE mutations. The mutation is a 12 nucleotide long deletion of a repetitive sequence in Exon 16 of the TFR2-α gene and was found by direct sequencing.

One finds at the location of the normal sequence 5′cccgccgtggcccaggccgtggcccagctcgcagggcagctcctc3′ (Sequence No. 2) 5′cccgccgtggcccagctcgcagggcagctcctc3′ (Sequence No. 3) as the mutated sequence.

It is present in the homozygote state and is associated with the phenotype which is inherited in the investigated family.

It was not present in 100 normal chromosomes.

As biological samples, blood, tissue biopsies, mouth interior scrapings, amniotic fluid and the like can be used, whereby the biological sample, depending upon the choice of the known investigation processes, can be subjected to also known pretreatments.

The object is also achieved in that a biological sample is analyzed for nucleic acids which code for a TFR2 gene product with an amino acid sequence Pro Ala Val Ala Gin Leu Ala Gly Gin Leu Leu (Sequence No. 5) instead of the amino sequence Pro Ala Val Ala Gin Ala Val Ala Gin Leu Ala Gly Gin Leu Leu (Sequence No. 4) or for the presence of a TFR2 gene product with an amino acid sequence Pro Ala Val Ala Gin Leu Ala Gly Gin Leu Leu (Sequence No. 5) instead of the amino acid sequence Pro Ala Val Ala Gin Ala Val Ala Gin Leu Ala Gly Gin Leu Leu (Sequence No. 4).

Especially the sample is analyzed for the presence of nucleic acids which code for a TFR2-α gene product with an amino acid sequence leucine-alanine-glycine-glutamine for the amino acid 594-597 or for the presence of a TFR2-α gene product with an amino acid sequence leucine-alanine-glycine-glutamine for the amino acids 594-597.

One finds in place of the normal amino acid sequence Pro Ala Val Ala Gin Ala Val Ala Gin Leu Ala Gly Gin Leu Leu (Sequence No. 4), the sequence Pro Ala Val Ala Gin Leu Ala Gly Gin Leu Leu (Sequence No. 5).

If the biological sample is also analyzed for the presence of nucleic acids whose TFR2 gene does not have the mentioned mutation or the last-mentioned amino acid sequence, it permits detection as to whether the mutation which is present is homozygote or heterozygote.

The analysis can be carried out in a known manner by sequencing the nucleic acid contained in the biological sample.

Or, however, the nucleic acids of the biological sample are brought into contact with at least one probe which is sensitized with nucleic acid of a biological sample in a region of the nucleotide sequence 5′cccgccgtggcccaggccgtggcccagctcgcagggcagctcctc3′ (Sequence No. 3) in Exon 16 of the TFR2 cDNA sequence, for hybridization and testing whether corresponding hybridization products are present.

Especially the nucleic acids of the biological sample are brought into contact with a probe which is capable of hybridization with a region of these nucleic acids which correspond to a region in Exon 16 of the TFR2-α cDNA sequence in the region of the nucleotide 1780-1791 when a deletion of the nucleotide 1780-1791 is present and it is then tested whether corresponding hybridization products are present.

If the nucleic acids of the biological specimen are brought into contact with at least one probe which is capable of hybridization with a region of these nucleic acids which contain the nucleotide sequence 5′cccgccgtggcccaggccgtggcccagctcgcagggcagctcctc3′ (Sequence No. 2), or with a region of these nucleic acids that corresponds to a region in Exon 16 of the TFR-2α cDNA sequence in the region of the nucleotide 1780-1791, and is then tested whether corresponding hybridization products are present, it can be established whether the mutation is present as a homozygote or heterozygote.

At the present time, a number of processes for the analysis of mutations are used, among them RFLP (restriction fragment length polymorphism) PCR-SSP(PCR with sequence specific primers), allele specific oligo nucleotide hybridization, SSCP (single-strand conformation polymorphism, DGGE (denaturing gradient gel electrophoresis), OLA (oligonucleotide ligation assay), real time fluorescence PCR and DNA sequencing. When RNA is analyzed, NASBA™ can be used (nucleic acid sequence-based amplification) Nature (1991) March 7; 350 (6313): 91-2, Compton J.: developed by Cangene Corporation, Mississauga, Ontario, Canada) or the RNA can be analyzed by means of rewriting with reverse transcription in cDNA, then amplified with PCR and then characterized. All of th se known analysis processes can be used for the diagnosis process according to the invention.

The probe of the invention for the diagnosis of haemochromatosis is capable of hybridization with nucleic acids of a biological sample in a range with contains the nucleotide sequence 5′cccgccgfggcccagctcgcagggcagctcctc3′ (Sequence No. 3) in Exon 16 of the TFR2 cDNA sequence.

A probe according to the invention is capable of hybridizing with nucleic acids of a biological sample which corresponds to a region in Exon 16 of the TFR2-α cDNA sequence corresponding to the regions of the nucleotide 1780-1791 when a deletion of the nucleotide 1780-1791 is present.

Below the family in which the new mutation has been found and the related investigations are reported upon.

A 32 year old man with a previously covered illness with iron overloading was newly evaluated. The clinical history showed neither alcohol consumption nor blood transfusion or extraordinary oral iron intake.

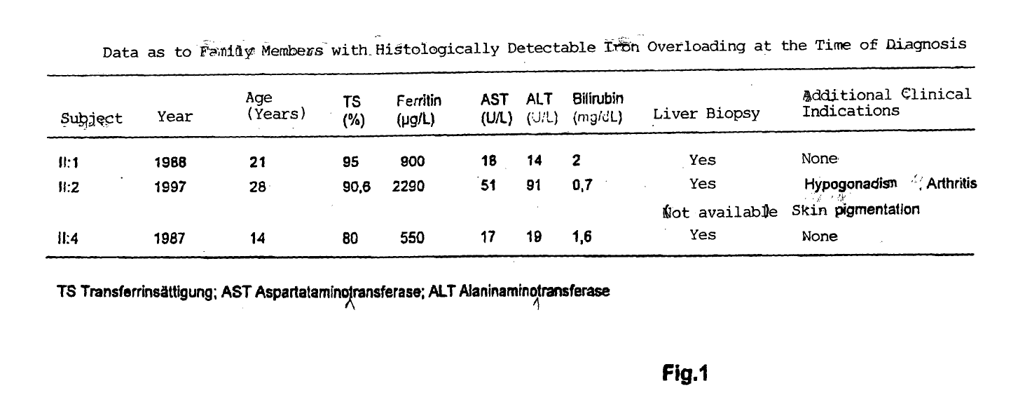

At the age of 16 years, in a biochemical analysis of sclerenicterus, an elevated serum level of bilirubin (2.8 mg/dL, primarily unbound) and iron (230 μg/dL) were found. An extended hematological study eliminated the presence of various of compensated iron-storing anemias and gave a Gilbert syndrome diagnosis on a clinical basis. At the time the serum ferritin level of 520 μg/L and the transferritin saturation was 76%. At the age of 21 years, a liver biopsy was carried out because of the continuing abnormalities of the biochemical parameters (FIG. 1). While a haemachromatosis diagnosis was made, therapy with phlebotomy was not commenced but the patient was treated for 4 years with intravenous deferoxamine (1 gram, 3 days a week by an infusion pump). At the end of the treatment the serum ferritin level was 553 μg/L. In the following year there was no treatment while the serum ferritin level was continuously >1000 μg/L.

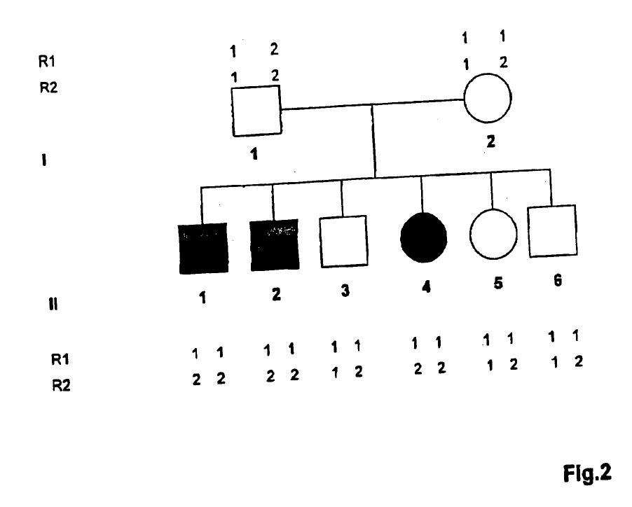

Both parents derived from many generations of northern Italians and were not symptomatic. The review of the clinical documents of family members showed that two siblings had been previously diagnosed with iron overloading. The family tree is given in FIG. 2. The ill family members have been shown by a solid symbol. An interfamilial segregation of two sequence repeats (R1 and R2) in the TFR2 gene, which have the identical haplo type, were found in the ill siblings.

EDTA blood samples were taken for the genetic analysis. DNA was recovered by conventional phenol-chloroform-extraction (Sambrook J., Frisch E., Maniatis T, Molecular Cloning: a laboratory manual, Cold Spring Harbor, N.Y.: Cold Sring Harbor Laboratory Press 1989). Using 12.5 pmol primer and 0.5 U Taq polymerase, a PCR (polymerase gene reaction) was carried out in a thermocycling apparatus (Perker Elmer, Shelton, Conn.) for 30 to 35 cycles in a total amount of 50 μL.

C282Y and H63D mutations in HFE were analyzed based upon amplified genomic DNA using PCR based tests and digestion with restriction enzymes Rsa I and Mbo I (New England Biolabs, Berkeley, Mass.) is described (Carella M, D'Ambrosio L, Totaro A, Grifa A, Valentino M A, Piperno A, Girelli D, Roetto A, Franco B, Gasparini P, Camaschella C. Mutation Analysis of HLA-H Gene in Italian Hernochromatosis Patients. Am J Hum Genet 1997; 60:828-32).

At the HH type 2 and type 3 loci respective linkage analyses were carried out by interfamilial segregation of microsatellite marker alleles of the chromosomes 1q and 7q22 as previously described. The microsatellite marker for the left of chromosome 1q was D1S2344, D1S442, D1S1156, GATA13C08 and D1S498 (Roetto A., Totaro A., Cazzola M, Cicilano M., Bosio S., D'Ascola G., Carella M., Zelante L., Kelly A. L., Cox T. M., Gasparini P., Camaschella C., The Juvenile Hemochromatosis Locus Maps to Chromosome 1q., Am J Hum Genet 1999, 64:1388-1391). The microsatellite marker for chromosome 7q was D7S651, D7S2498, D7S662, D7S477, D7S1S88 and 2 TFR2 intragene repetitions (R1 and R2) (Camaschella C., Roetto A., Cali A., De Gobbi M., Garozzo G., Carella M., Majorano N., Totaro A., Gasparini P., The Gene Encoding Transferrin Receptor 2 is Mutated in a New Type of Hemochromatosis Mapping to 7q22., Nat Genet 2000; 25:14-15;: Roetto A., Totaro A., Piperno A., Piga A., Longo F., Garozzo G., Cali A., De Gobbi M., Gasparini P., Camaschella C., New Mutations Inacativating Transferrin Receptor 2 in Hemochromatosis Type 3., Blood 2001; 97:2555-2560). Primer was produced corresponding to the data bank sequences.

During the sequencing of purified PCR product is carried out by use of the thermosequenase Cy 5.5 sequencing kit (Amersham-Pharmacia, Uppsala, Sweden) and a Seq 4×4 apparatus (Amersham-Pharmacia Biotech, Uppsala, Sweden).

Acrylamide gel electrophoresis was carried out in a gene-Phorelectrophoresis system (Amersham Pharmacia Biotech) with 6% finished acrylamide gel. An analysis of the (TA)₇ promotor insertion in bilirubin uradine phosphate glucocuronosyltransferase (UGT)1-gene was carried out as described (Bosma P. J., Chowdhury J. R., Bakker C., Gantla S., de Boer A., Oostra B. A., Linhout D., Tytgat G. N. J., Jansen P. L. M., Elferink R. P. J. O., Chowdhury J. R., The Genetic Basis of the Reduced Expression of Bilirubin UDP-Glucuronosyltransferase 1 in Gilbert's Syndrome, N. Engl. J. Med 1995, 333:1171-1175).

The controls were 100 healthy blood donors from the same geographic region as the family.

### Results

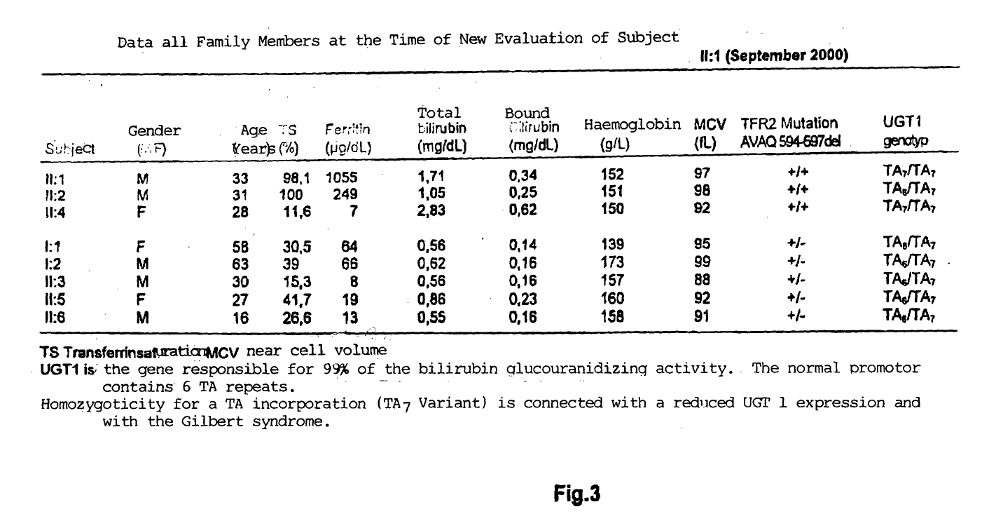

The first liver biopsy samples of the test individual were found and once again worked up. The histopathological evaluation showed normal lobe-forming structures, no significant fibrosis and hepatocellular hemosiderin deposits of the third degree according to Scheuer et al (Scheuer P. J., William R., Muir A. R., Hepatic Pathology in Relatives of Patients with Hemochromatosis, J. Pathol. Bacteriol 1962:84:53-64). Biochemical data of the test individual are reproduced in FIG. 3 at the time of reevaluation. Upon body investigation, the edge of the liver was sensitive to touch and 3 cm under the right rib edge was palpable. There was no noticeable skin pigmentation. There were neither clinical nor biochemical indications of either diabetes mellitus or hypogonadism. Electrocardiography and echo cardiography were normal. Since the patient had undergone no appropriate therapy for a long period of time, a new biopsy for determining the status of the liver disorder was carried out. The histopathological evaluation indicated a normal lobular structure, only a slight degree of fibrosis and hepatocellular hemosiderin deposits of the fourth degree according to Scheuer et al. The iron concentration in the liver was 358 μmol/g dry weight and the iron index in the liver (ratio of iron concentration) [μmol/g dry weight] in the liver to age [years] was 11.2. Then a phlebotomy therapy was commenced and after removal of 8.75 g of iron, a serum ferritin level (46 μg/L) was reached which was below the normal value by contrast with which the transferrin saturation remained increased (100%).

The 28 year old sister (II:4) was at the age of 14 years first evaluated based upon a sclerenicterus. At this time a liver biopsy was made which was repeated and then evaluated again. The clinical history showed that the patient, several months after the liver biopsy, developed an eating disorder with progressive major iron loss. Anorexia nervosa was diagnosed and she was treated until the age 20 with psychotherapy in which the eating disorder improved. As her weight stabilized above 50 kg, she became a regular blood donor. After eight blood donations (each 350 mL) the hemoglobin level reduced to 12 g/dL. In the same time period she underwent esophagogastro-duodenoscopy because she had dyspeptic symptoms. The endoscopy confirmed the presence of a chronic gastritis in the prepyloric section of the stomach because of a heliobacter pylori infection. The biochemical data of the patient is reproduced at the time of reevaluation together with those of all other family members in FIG. 3.

### Molecular Studies

None of the known HFE mutations were found in any of the family members. Juvenile haemochromatosis was excluded since the ill siblings had different 1q haplotypes in the HH type 2 critical region.

Intrafamilial segregation of marker alleles of the chromosome 7q matched with the combination of the HH type 3 locus; the three ill siblings were haplotype identical and their 7q haplo types were different from those of each of the non-ill siblings. The results are reproduced as to the sequence repetitions R1 and R2 in the TFR2 gene, in FIG. 2.

A mutation analysis of the entire TFR1-α coding sequence (18 Exons) and the Exon/Intron boundaries of the genomic DNA of patients II:2 and II:4 gave a single identical homozygote mutation in both subjects. The mutation was a 12 nucleotide-long deletion in a 12-nucleotide-long repetition in Exon 16 at the position 1780-1791. This deletion contributed to a 4-amino acid (AVAQ) long deletion at the position 594-597 in the protein.

Since a PCR fragment shorter by 12 nucleotides was obtained after amplification of Exon 16, the segregation of the AVAQ mutation was analyzed by acrylamide gel electrophoresis. This analysis showed that in the family, the segregation of the deletion was followed by the illness; it was present in the homozygote state in the test individuals II:1, II:2 and II:4 and in the heterozygote state in all other family members (FIG. 3). The deletion was not found in 100 healthy controls.

Because of the continuously elevated bilirubin level in many of the family members, the (TA) repeat insertion in the UGT1 gene promotor was analyzed. A (TA)₇(TA)₇ pattern showed in family members with hyperbilirubinemia (II:1 and II:4) in harmony with the diagnosis, a Gilbert syndrome.

The following example describes possible embodiments of a method according to the invention for the genetic diagnosis of haemochromatosis.

### DNA Isolation and Amplification:

DNA is isolated from an anticoagulated blood using conventional extraction processes (A Simple Salting Out Procedure for Extracting DNA from Human Nucleated Cells” Miller et al., 1988) or commercially available reagents (GenXtract DNA Extraction System, ViennaLab, Vienna, AT). The Exon 16 sequence of the TFR2-α gene was amplified in a PCR reaction using the primers 5′-cccagcgtccaccctgtcctggc-3′ (Sequence No. 6) and 5′-ctggattgccagagaggacc-3′ (Sequence No. 7). Each primer was used with a final concentration of 25 pM.

A thermocycling program with 30 cycles (94° C. for 15 seconds, 58° C. for 30 seconds and 72° C. for 30 seconds) was carried out with the GeneAmp PCR System 2400 (PE Biosystems, Foster City, CA).

### Sequencing

The PCR products were purified with a “Centricon-100” solution in accordance with the instruction of the manufacturer. The amplified and purified DNA was quantified by UV spectrophotometry and then sequenced in accordance with strict adherence to the “BigDye Terminator Cycle Sequencing Ready ReactionKit—with AmpliTaq DNA Polymerase, FS” (PE Applied Biosystems) for ABI prism instrumentation (PE Applied Biosystems).

1. A method of diagnosing haemochromatosis, characterized in that a biological sample is analyzed for the presence of the nucleotide sequence 5′-cccgccgtggcccagctcgcagggcagctcctc-3′ (Sequence No. 3) instead of nucleotide sequence 5′-cccgccgtggcccaggccgtggcccagctcgcagggcagctcctc-3′ (Sequence No. 2) for a 12 nucleotide long deletion in Exon 16 of the TFR2 cDNA sequence.

2. The method according to claim 1, characterized in that a biological sample is analyzed for the presence of a deletion of the nucleotides 1780-1791 in Exon 16 of the TFR2-α cDNA sequence (sequence No. 1).

3. The method of diagnosing haemachromatosis, characterized in that a biological sample is analyzed for the presence of nucleic acids which code for a TFR2 product with the amino acid sequence
Pro Ala Val Ala Gin Leu Ala Gly Gin Leu Leu (Sequence No. 5) instead of the amino sequence Pro Ala Val Ala Gin Ala Val Ala Gin Leu Ala Gly Gin Leu Leu (Sequence No. 4) or for the presence of a TFR2-α product with an amino acid sequence Pro Ala Val Ala Gin Leu Ala Gly Gin Leu Leu (Sequence No. 5) instead of the amino acid sequence Pro Ala Val Ala Gin Ala Val Ala Gin Leu Ala Gly Gin Leu Leu (Sequence No. 4).

4. The method according to claim 3, characterized in that a biological sample is analyzed for the presence of nucleic acids which code for a TFR2-α product with an amino acid sequence leucine-alanine-glycine-glutamine for the amino acids 594-597 or for the presence of TFR2-α gene product with an amino acid sequence leucine-alanine-glycine-glutamine for the amino acids 594-597.

5. The method according to one of the preceding claims, characterized in that the biological sample is also analyzed for the presence of nucleic acids whose TFR2 gene does not have the mentioned mutation or the mentioned altered amino acid sequence.

6. The method according to one of the preceding claims, characterized in that the analysis is carried out in a manner known per se by sequencing the nucleic acid obtained from the biological sample.

7. The method according to claim 1, characterized in that nucleic acid from the biological sample is brought into contact with at least one probe having the capacity for hybridizing with a region of these nucleic acids which contains the nucleotide sequence 5′ cccgccgtggcccagctcgcagggcagctcctc 3′ (Sequence No. 3) and that a test is made whether corresponding hybridization products are present.

8. The method according to claims 2 and 7, characterized in that nucleic acids from the biological sample are brought into contact with at least one probe which is capable of hybridizing with a region of these nucleic acids which correspond to a region in Exon 16 of the TFR2-α cDNA sequence in the region of the nucleotide 1780-1791 when a deletion of the nucleotide 1780-1791 is present and it is tested whether corresponding hybridization products are present.

9. The method according to claim 7, characterized in that nucleic acids of the biological sample are brought into contact with at least one probe which is capable of hybridizing with a region of these nucleic acids which contain the nucleotide sequence 5′-cccgccgtggcccaggccgtggcccagctcgcagggcagctcctc-3′ (Sequence No. 2) and it is tested whether corresponding hybridization products are present.

10. The method according to claim 8, characterized in that nucleic acids of the biological sample are brought into contact with at least one probe which is capable of hybridizing with a region of these nucleic acids which corresponds to a region in Exon 16 of the TFR2-α cDNA sequence in the region of the nucleotide 1780-1791 when there is no mutation present in the region of the nucleotide 1780-1791 and it is tested whether corresponding hybridization products are present.

11. A probe for the diagnosis of haemochromatosis, characterized in that the probe is capable of hybridization with nucleic acids of a biological sample in a region which contains the nucleic data sequence 5′-cccgccgtggcccagctcgcagggcagctcctc-3′ (Sequence No. 3) in Exon 16 of the TFR2 cDNA sequence.

12. The probe according to claim 11, characterized in that the probe is capable of hybridization with nucleic acids of a biological sample in a region corresponding to a region in Exon 16 of TFR2-α cDNA sequence in the region with nucleotides 1780-1791 when a deletion is present of the nucleotide 1780-1791.
